(19) United States
(12) Patent Application Publication
(10) Pub. No.: US 2016/0262928 A1
(43) Pub. Date: Sep. 15, 2016

(54) Reversible Thermal Pack

(71) Applicant: Joseph M. Carpentier, Broomfield, CO (US)

(72) Inventor: Joseph M. Carpentier, Broomfield, CO (US)

(21) Appl. No.: 15/067,447

(22) Filed: Mar. 11, 2016

Related U.S. Application Data
(63) Continuation-in-part of application No. 29/521,726, filed on Mar. 26, 2015.
(60) Provisional application No. 62/132,051, filed on Mar. 12, 2015.
(60) Provisional application No. 62/188,496, filed on Jul. 3, 2015.

(51) Int. Cl. A61F 7/02; A61F 7/10

(52) U.S. Cl. CPC A61F 7/02; A61F 7/10; A61F 2007/0228; A61F 2007/0279; A61F 2007/108

(57) ABSTRACT

An insulated outer case is shaped like a clam shell, preferably with a fabric hinge. In a top half is an ice bag or gel pack. In a lower half is an optional pocket holding a long securing wrap and anchored short straps for encircling a limb. In a closed mode the ice bag is surrounded by two insulated clam shell halves secured together by a zipper. In an open mode the straps can be adjusted and the bag filled with ice (or water to be heated). Then the halves are reversed and zippered into the reverse mode for application. A combo pack embodiment adds a drinking water bladder to the bottom half.

### CROSS REFERENCE APPLICATIONS

The co-pending design patent application Ser. No. is 29/521,726 filed Mar. 26, 2015. Provisional application No. 62/132,051 was filed Mar. 12, 2015 and Provisional application No. 62/188,496 was filed Jul. 3, 2015. Priority is claimed from all three applications, and all three applications are incorporated herein by reference in their entirety.

### FIELD OF INVENTION

The present invention relates to providing a cold or hot pack to an injured part of the human body with straps, and providing a reversible/carrying mode to insulate and transport the cold or hot pack.

### BACKGROUND OF THE INVENTION

There are presently known a large number of devices used to apply heat or cold to parts of the human body. One set of such prior art devices relies on hot or cold water to supply the therapeutic source of heat or cold. While liquid water at different temperatures is generally conveniently available, the heat or cold carrying capacity of water is limited when no phase change is involved.

Another set of such prior art devices relies on gel packs as a source of heat or cold. While some such packs have more heat or cold carrying capacity than water, they are relatively expensive and their capacity is still limited. U.S. Pat. No. 4,899,749 discloses thermal vascular dilating device and method employing a cylindrically shaped water-tight container with a threaded spout and cap opening for filling the container. Hook and loop fastener straps are used to secure the device to the arm of the human body.

For applying cold to (removing heat from) the human body, ice has long been recognized as having superior cold carrying capacity when compared with all simple liquids and many other substance that are capable of undergoing a phase change. The phase change energy or ice turning to water is very high, and ice is therefore a very effective source of cold. Ice is also inexpensive and readily available in a variety of settings.

U.S. Pat. No. 4,753,240 discloses a device for immobilizing and applying heat or cold to a body part. It includes one or more pouches for enclosing ice packs or heat packs. A complex arrangement of straps allows these packs to be positioned in various relationships to an arm and shoulder. This device assumes that a certain degree of immobility is desirable or necessary, and, accordingly, includes metal stays for this purpose. However, on many occasions, people with minor body part injures wish to remain as active as possible, while still deriving the beneficial effects of cold therapy, and this device is inappropriate to their needs.

U.S. Pat. No. 4,628,932 discloses an ice pack specialized for use on the human knee. It includes multiple compartments for receiving the ice and a hole intended to be aligned with the patella to keep it from being subjected to the cooling effect of the ice. The multiple compartments are accessed through a zipper, and the pack is secured to the leg with a pattern of hook and loop fattener strips. This pack is highly specialized for the knee and is fastened in a way that presumes some immobility on the part of user.

The icing system of U.S. Pat. No. 5,215,080 has multiple compartments, an insulating layer of wetsuit rubber, including flanges, hook and loop straps, fasteners, and dividers in a unique geometry, and is designed to use resealable plastic sandwich and freezer bags as its ice containers. The multiple compartments allow varying amounts of ice to be put around different areas of the same body part, make it flexible, and permit a maximum of mobility on the part of the user. The wetsuit rubber that is used as an outer insulator, and in the flanges that adjoin the user's skin allow this icing system to use ice efficiently and permit maximum mobility of the user.

In U.S. Pat. No. 5,020,711 the holder for use with a thermos transfer pack, is made of cloth material, essentially rectangular in shape, open at one end, and slightly larger than the corresponding thermos pack. The thermos pack is slid into the holder and retained there by sealing with closing means. The holder/pack combination is placed on a portion of the user's body for the purpose of applying hot or cold treatment to the area. Elastic bands affixed at one end to the holder wrap around the portion of the body being treated and attach again to the holder by means of a hook and loop fastener system. In the event that the bands affixed to the holder are not of sufficient length to completely wrap around the treated area, additional elastic bands of greater length with hook and loop fasteners at each end are used and attached to either the holder or an elastic band.

Hook and loop strips are placed at the top and bottom of each holder for securing the holder by attaching to the corresponding hook and loop strips on the elastic bands. These hook and loop strips may also be used to combine more than one holder in an essentially modular system for applying treatment to a wider area.

U.S. Pat. No. 8,771,331 provides a multi-use, disposable cold therapy device that is easy to fill with a cooling agent and stably apply to the target area of the body. It applies focused compression to the desired target location using elastic wings.

What is needed in the art is an insulated carrying case that is integral to the cold/hot pack. An optional drinking water bladder could be included in the carrying case. The present invention provides these features in a reversible clam shell style carry case about the size of a catcher's mit. The cases can be daisy chained also.

### SUMMARY OF THE INVENTION

The main aspect of the present invention is to provide an insulated carry case for a hot/cold pouch, wherein the carry case can be reversed for travel or opened for application of the hot/cold pouch to the injured area through a thin fabric.

Another aspect of the present invention is to provide a clam shell shape for the carry case with a hinge joining the two halves and a zipper used to close the carry case.

Another aspect of the present invention is to provide a pair of crisscross straps in the carry case and an optional long elastic wrap as well.

Another aspect of the present invention is to provide a part to remove the (rubber) pouch from the carry case for refill purposes.

Another aspect of the present invention is to provide a mesh pocket in the carry case.

Another aspect of the present invention is to provide a handle on the carry case.

Another aspect of the present invention is to provide a second embodiment having a water bottle pouch built integral into the opposing clam shell half, thus providing both an ice pack and a water bottle for the user. The ice pack can cool the drinking water.

Other aspects of this invention will appear from the following description and appended claims, reference being made to the accompanying drawings forming a part of this specification wherein like reference characters designate corresponding parts in the several views.

### BRIEF DESCRIPTION OF THE DRAWINGS

FIG. 1 is a left side elevation view of the pack in use in the reverse mode strapped on a knee.

FIG. 2 is a front elevation view of the pack of FIG. 1 in a closed mode.

FIG. 3 is a rear elevation view of the pack of FIG. 1 in a closed mode showing the carry handle.

FIG. 4 is a side mirrored elevation view of the pack of FIG. 1 in the closed mode.

FIG. 5 is a top plan view of the pack of FIG. 1 in the closed mode.

FIG. 6 is a bottom plan view of the pack of FIG. 1 in the closed mode.

FIG. 7 is a top perspective view of the pack of FIG. 1 in the closed mode.

FIG. 8 is a top perspective view of the pack of FIG. 1 in the open mode ready to be folded into the reverse mode for use.

FIG. 9 is a bottom plan view of the pack as shown in FIG. 8.

FIG. 10 is a left side elevation view of the pack as shown in FIG. 9.

FIG. 11 is a right side elevation view of the pack as shown in FIG. 9.

FIG. 12 is a front elevation view of the pack as shown in FIG. 9.

FIG. 13 is a rear elevation view of the pack as shown in FIG. 9.

FIG. 14 is a bottom perspective view the pack in the open mode.

FIG. 15 is a bottom plan view of the pack in the reverse mode ready for use showing the applicator side.

FIG. 16 is a top plan view of the pack ion the reverse mode showing the crisscross straps.

FIG. 17 is a front elevation view of the pack as shown in FIG. 15.

FIG. 18 is a rear elevation view of the pack as shown in FIG. 15.

FIG. 19 is a side mirrored view of the pack as shown in FIG. 15.

FIG. 20 is a bottom perspective view of the pack as shown in FIG. 15.

FIG. 21 is a top perspective view of the pack of FIG. 5 showing the lid of the rubber water bottle extended through the insulated top cover.

FIG. 22 is a side perspective view of the pack of FIG. 21 in transition to the reverse mode.

FIG. 23 is a top perspective view of the pack of FIG. 22 in the open mode.

FIG. 24 is a side perspective view of the pack of FIG. 23 in the reverse mode.

FIG. 25 is an exploded view of the pack in FIG. 24.

FIG. 26 is front elevation view of a daisy chain pair of packs strapped to a user's hips.

FIG. 27 is a side perspective view of a daisy chain pair of packs on a user's left shoulder.

FIG. 28 is a left side perspective view of a pack in use against a user's right hip.

FIG. 29 is a front perspective view of a pack in use against a user's right shoulder.

FIG. 30 is a side perspective view of a daisy chain pair of packs against a right knee.

FIG. 31 is a side perspective view of a pack against a right knee.

FIG. 32 is a tip perspective view of a combination pack and drinking water embodiment.

FIG. 33 is a top perspective view of the FIG. 32 embodiment in the reverse mode ready for application.

FIG. 34 is an exploded view of the embodiment shown in FIG. 32.

FIG. 35 is a top perspective view of a partially opened combo pack 3200.

FIG. 36 is a top perspective view of the combo pack 3200 ready for use in the closed mode.

Before explaining the disclosed embodiment of the present invention in detail, it is to be understood that the invention is not limited in its application to the details of the particular arrangement shown, since the invention is capable of other embodiments. Also, the terminology used herein is for the purpose of description and not of limitation.

### DETAILED DESCRIPTION OF THE DRAWINGS

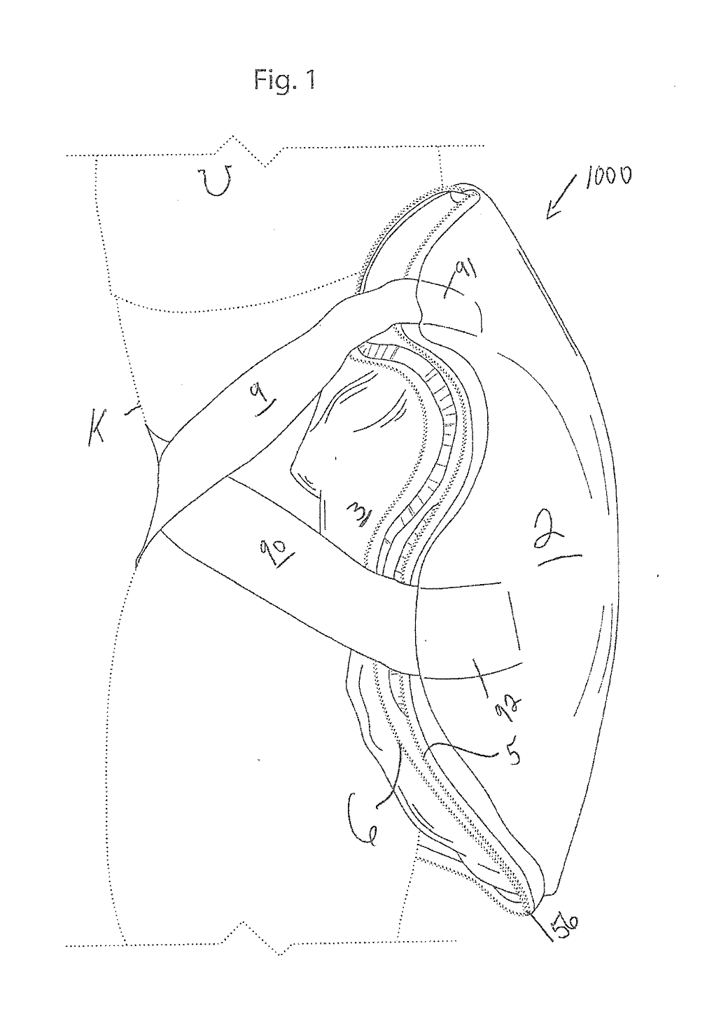

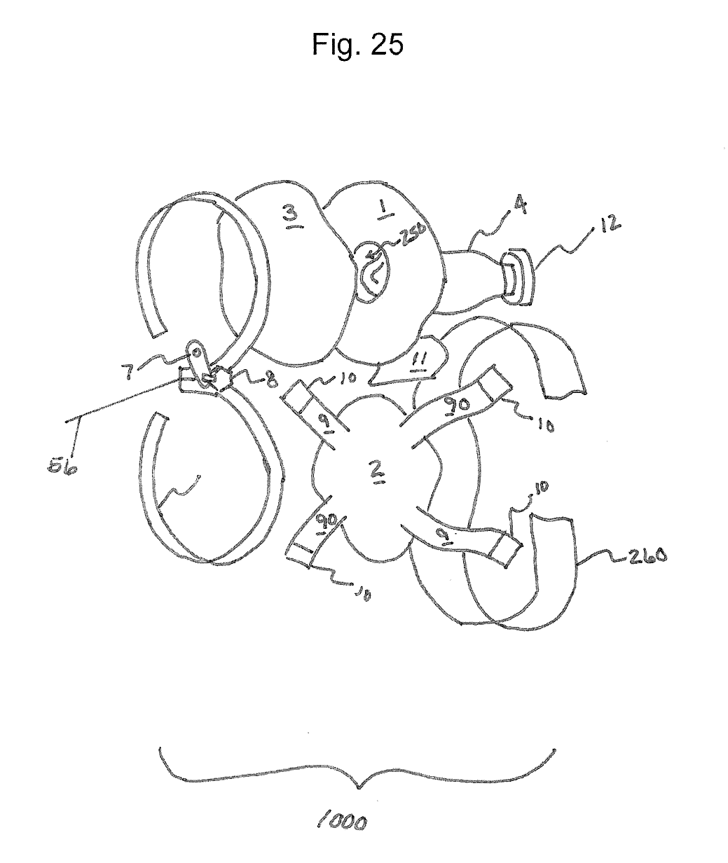

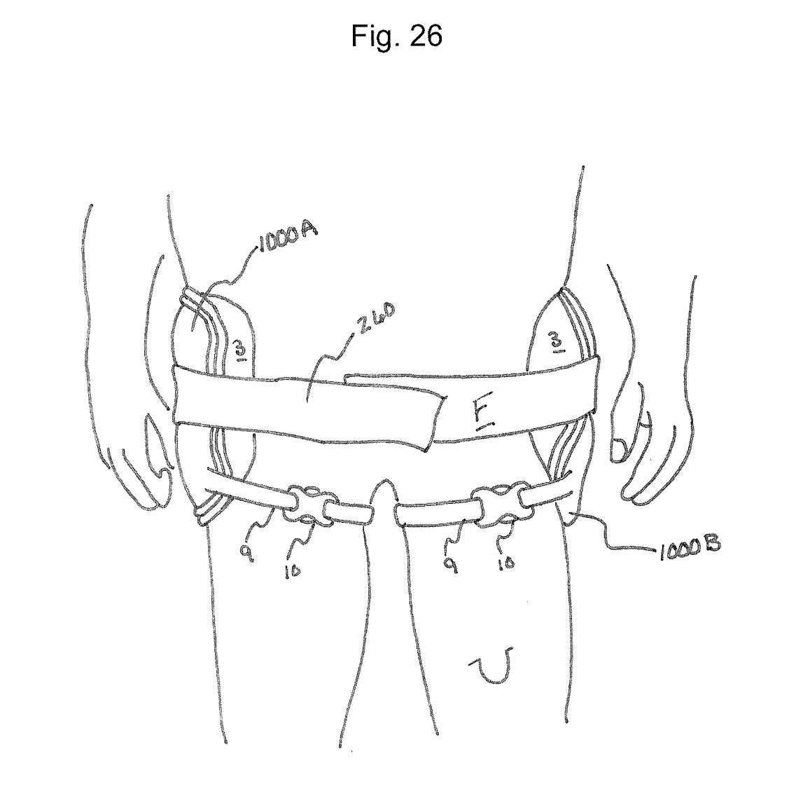

Referring first to FIG. 1 a thermal pack 1000 is in use against a knee K of user U. An application fabric 3 may be a thin non-insulating fabric such as a waterproof canvas. A rubber water bottle 4 may be full of ice or a gel pack, please refer to the exploded view of FIG. 25. A bottom half 2 of the clam shell design is an insulated fabric preferably of a foam construction. Straps 9, 90 are crisscrossed. They are anchored (via stitching) into the bottom half 2 at areas 91, 92. Buckles 10 can be seen in FIG. 25. Zipper 56 has tracks 5, 6. The zipper 56 has closed the top clam shell half 1 inside the thermal pack 1000. Equivalent closure means include hook and loop fastness, snaps and buttons. Clam shell half 1 is also insulated. Thus, a double insulated covering (1 and 2) keeps the ice (not shown) inside the water bottle 4 from melting too quickly. The straps can be pulled tightly through the buckles 10 in a known manner to provide compression on the knee if desired.

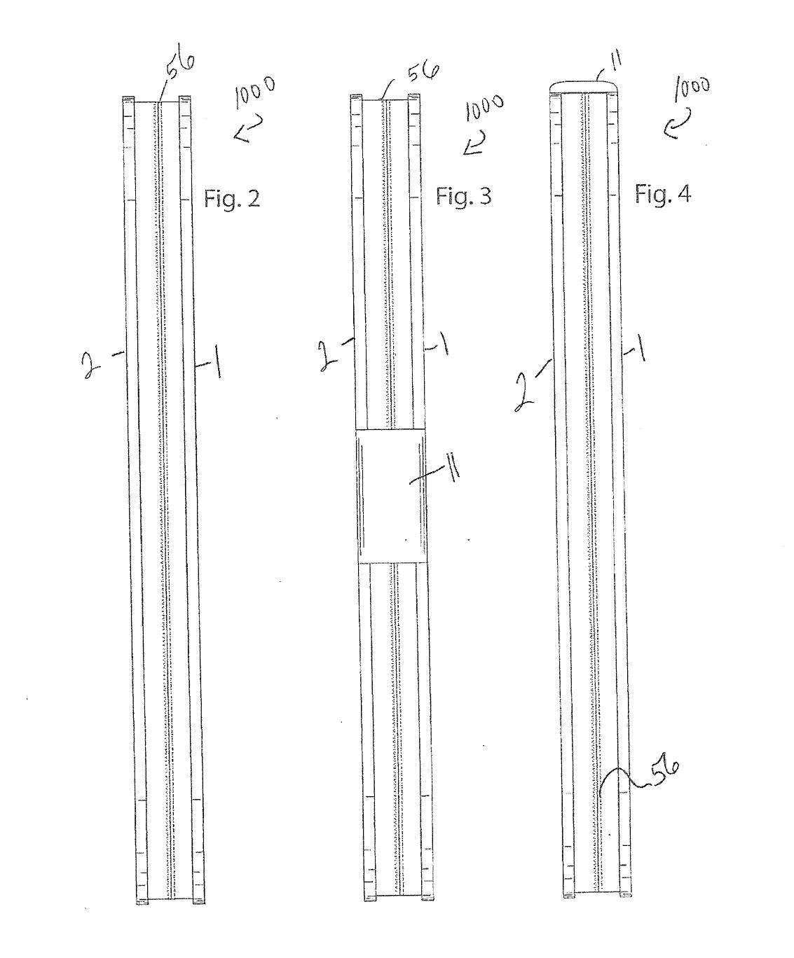

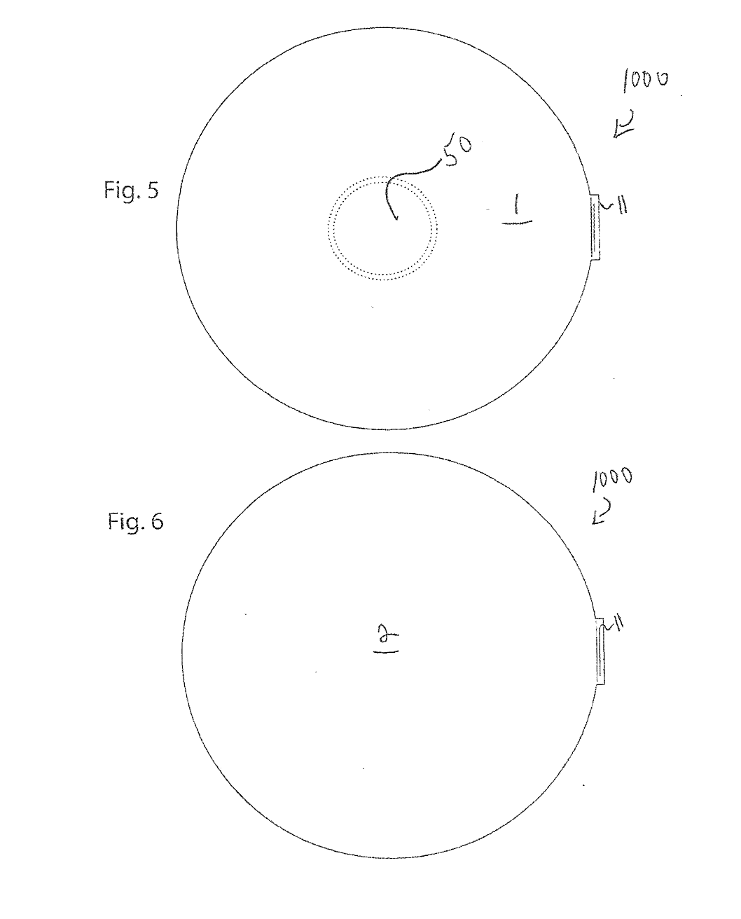

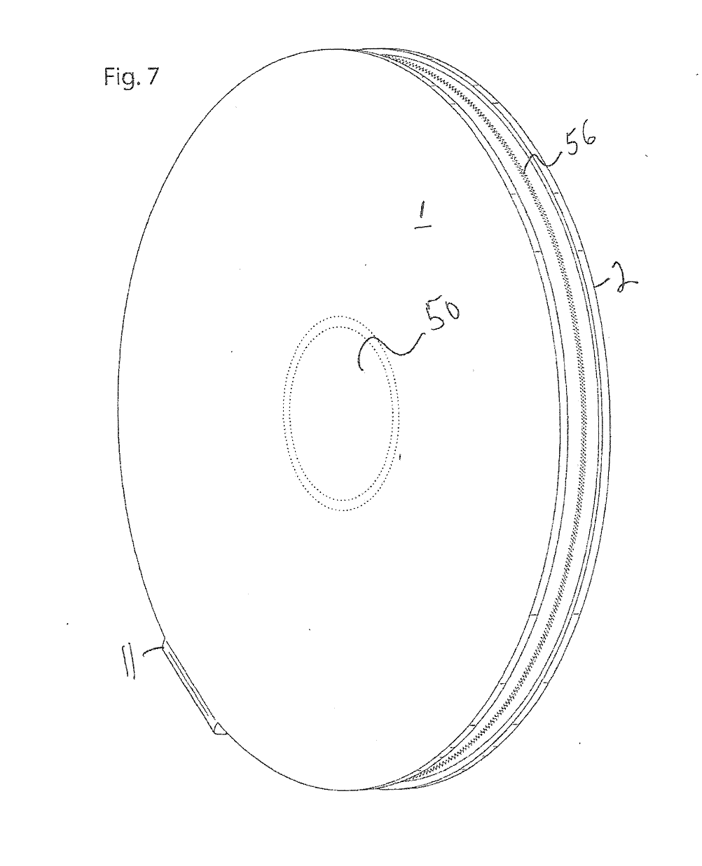

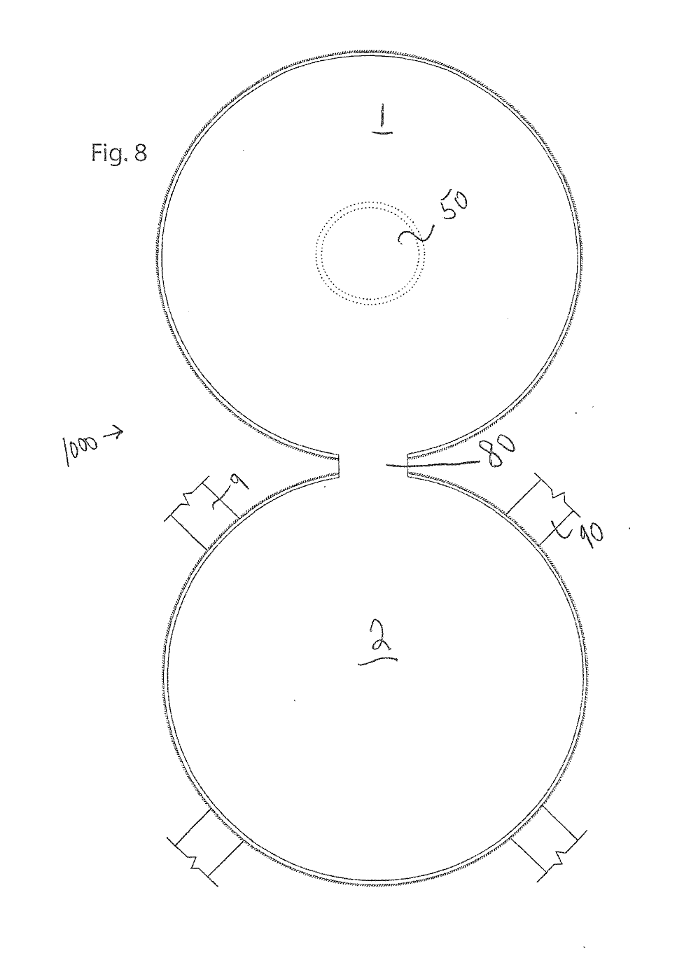

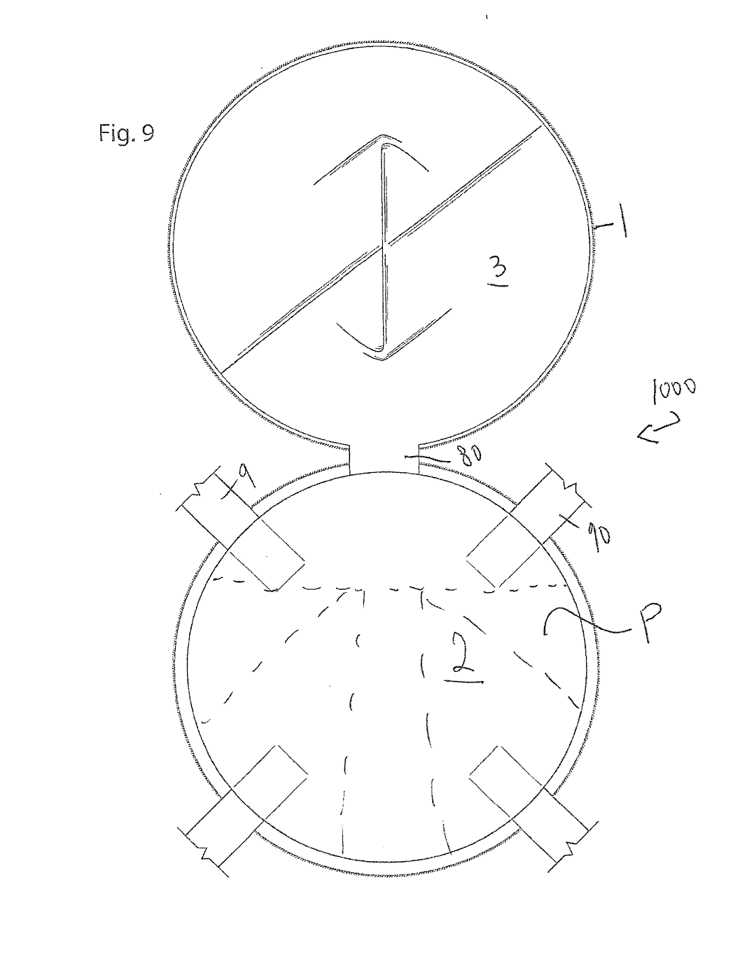

FIGS. 2-4 show the pack 1000 in the closed (transport) mode. FIGS. 5, 6 and 7 show a flap 50 that covers the lid 12 of eth water bottle and covers the filler hole 250 of clam shell half 1.

Referring next to FIG. 8 clam shell half 1 may be connected to bottom clam shell half 2 by a fabric hinge 80. This open mode shown serves to allow the user to pull out the straps 9. 90, adjust their lengths, fill the water bottle 4 with ice or fill it with water and microwave it for use as a hot pack. A gel pack could be substituted for the water bottle 4.

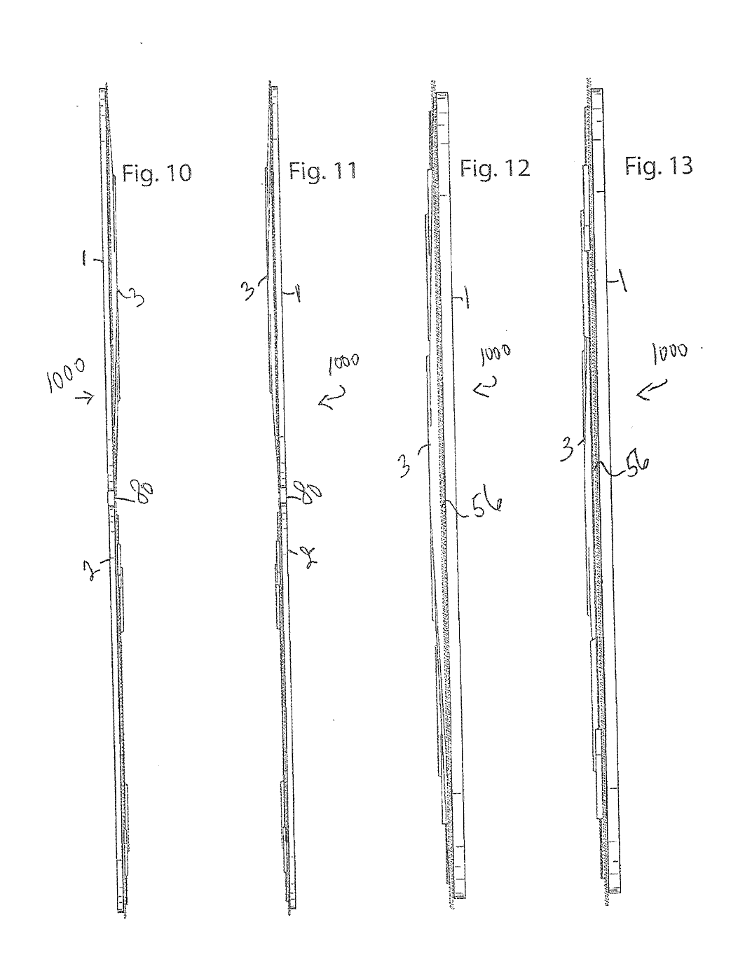

Referring next to FIG. 9 a mesh pocket P is sewn into the inside of clam shell half 2. This pocket P can store straps 9, 90 as well as a long wrap 260 as shown in FIG. 26, FIGS. 10-13 show various views in the open mode.

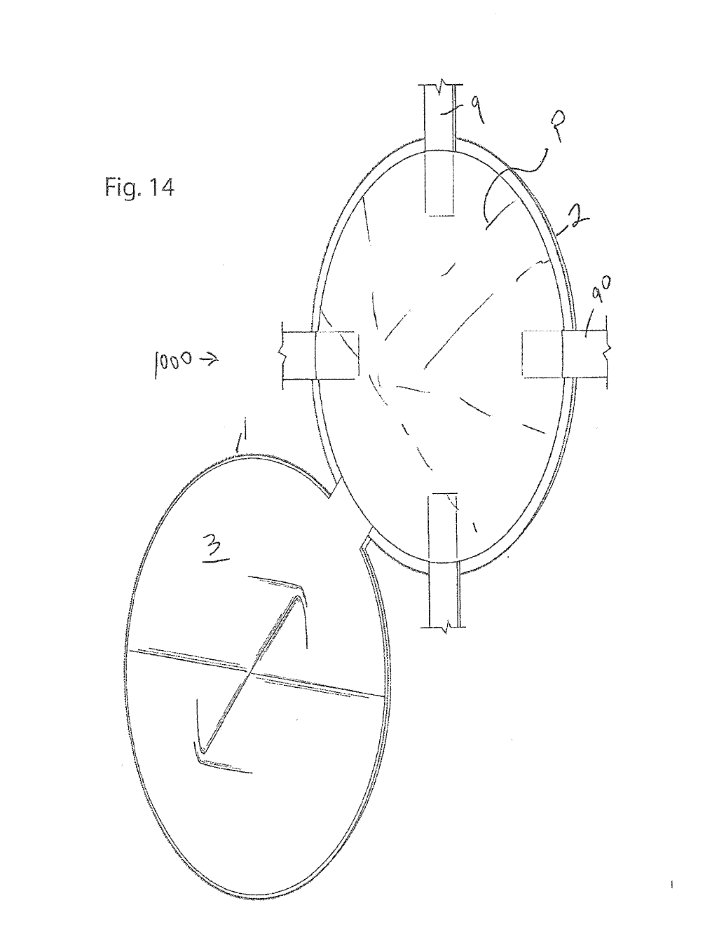

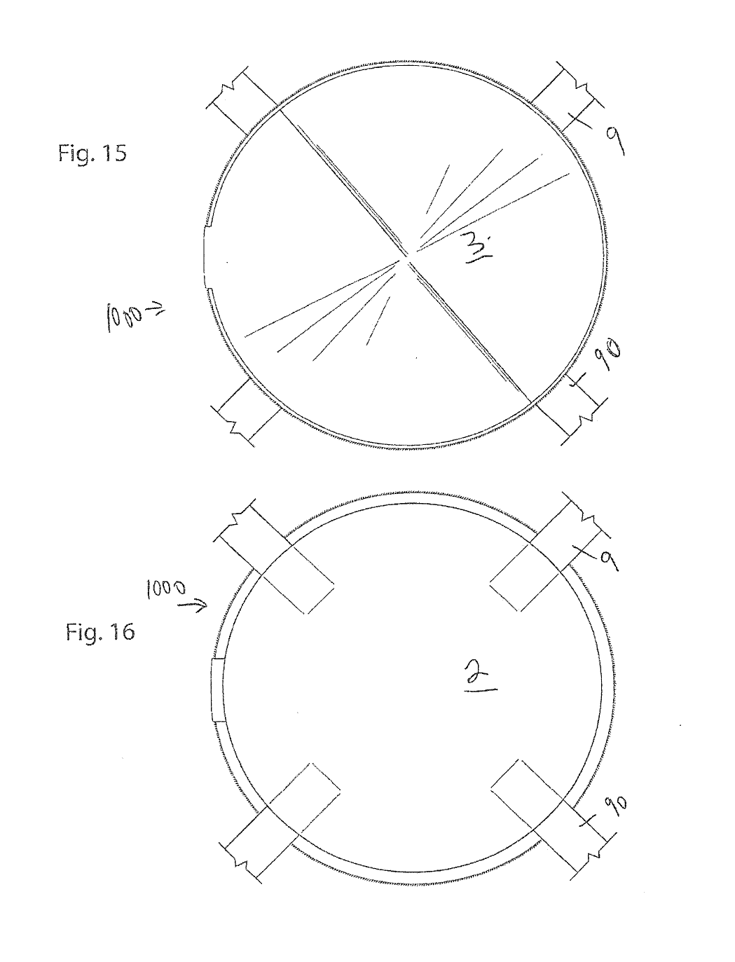

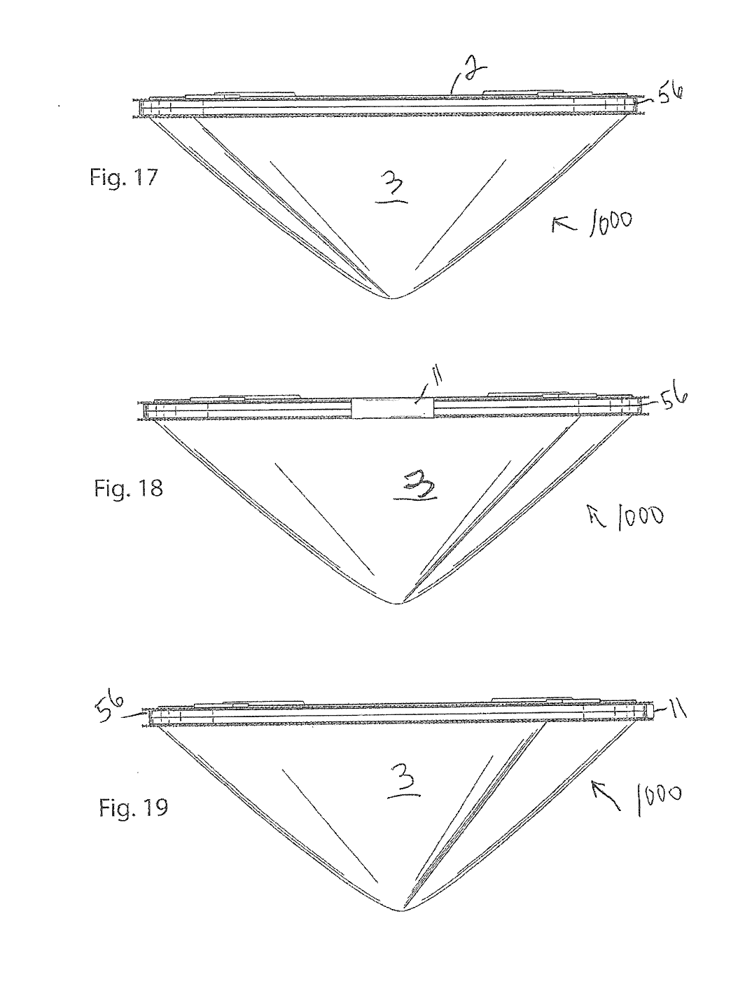

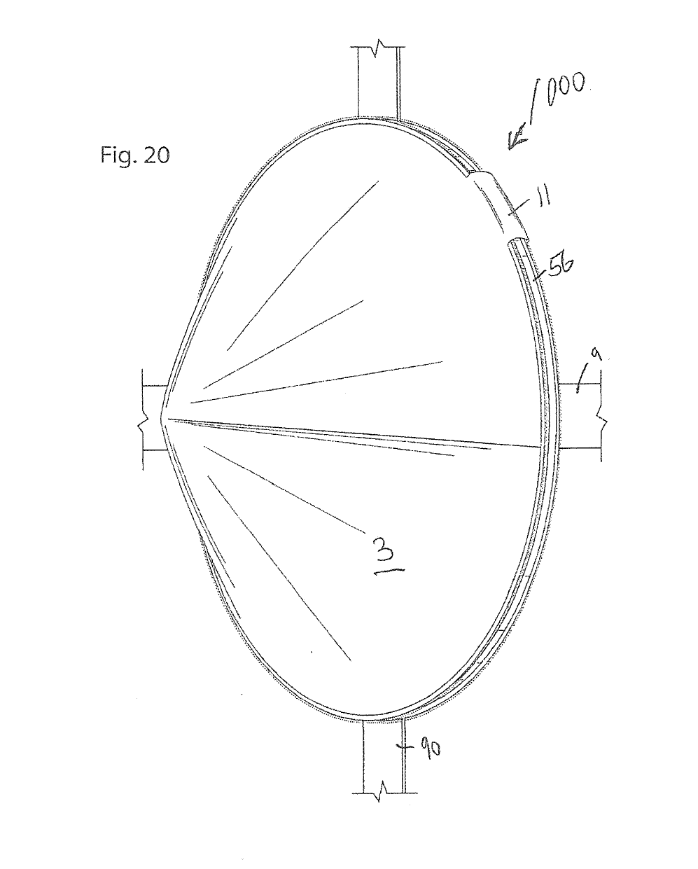

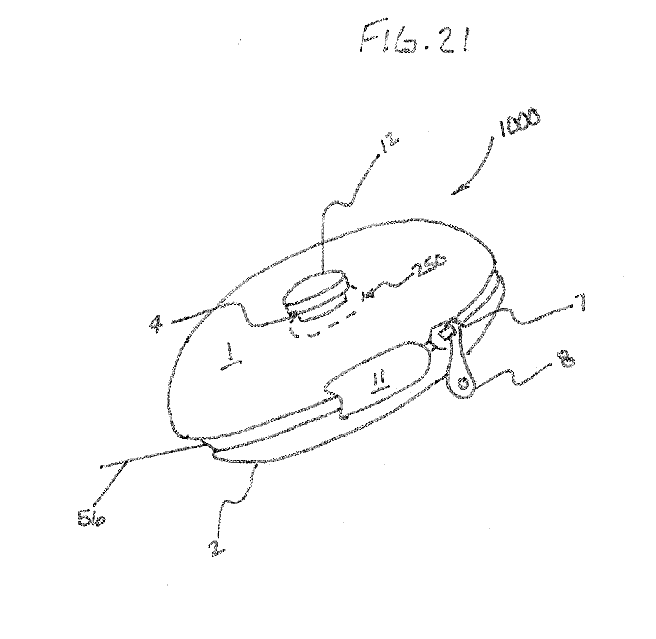

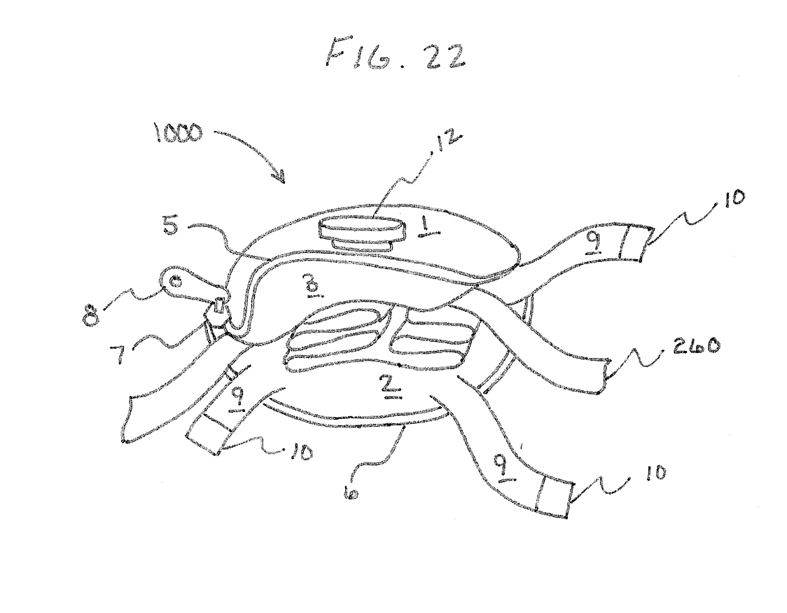

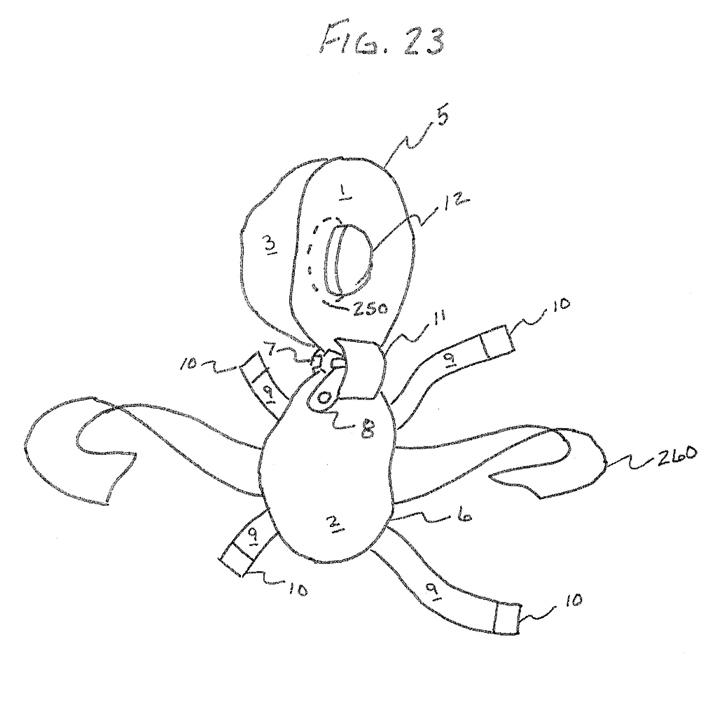

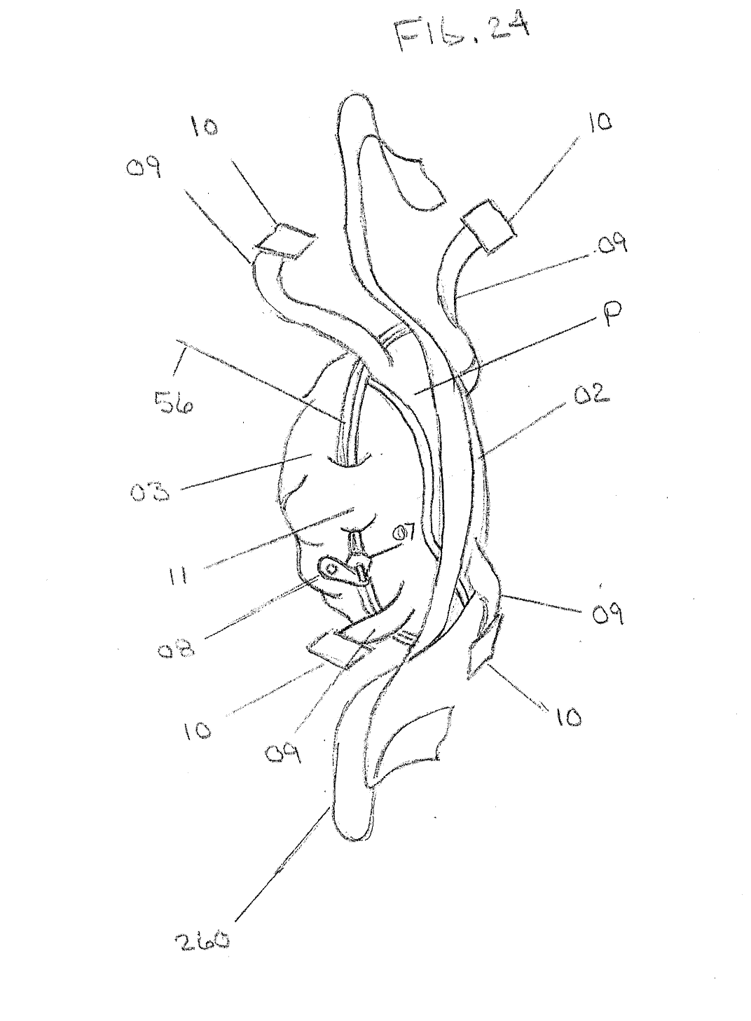

Referring next to FIG. 14 the open mode is shown getting ready to close halves 1, 2 and zipper the pack in the reverse mode shown in FIGS. 15-20.

Referring next to FIG. 21 the water bottle 4 and lid 12 are protruding from hole 250 in clam shell half 1. The zipper tab 8 is fastened to the zipper slider 7.

In FIG. 22 the user U is opening the zipper 56 to transition from a closed mode to the open mode shown in FIG. 23. In FIG. 24 the user is ready to apply the pack 1000 as shown in FIG. 1.

Referring next to FIG. 26 the user U has used identical packs 1000A, 1000B to treat both hips. A long wrap with a hook and loop fastener F has secured both packs 1000A, 1000B to the user's hips. Smaller straps and buckles 9, 10 further secure the packs to the legs.

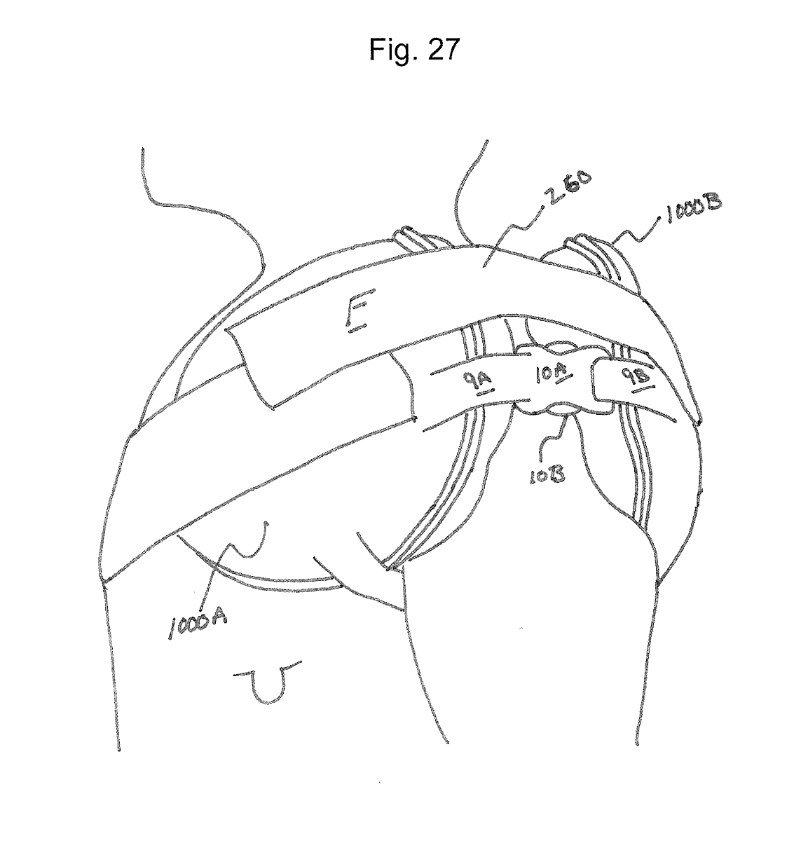

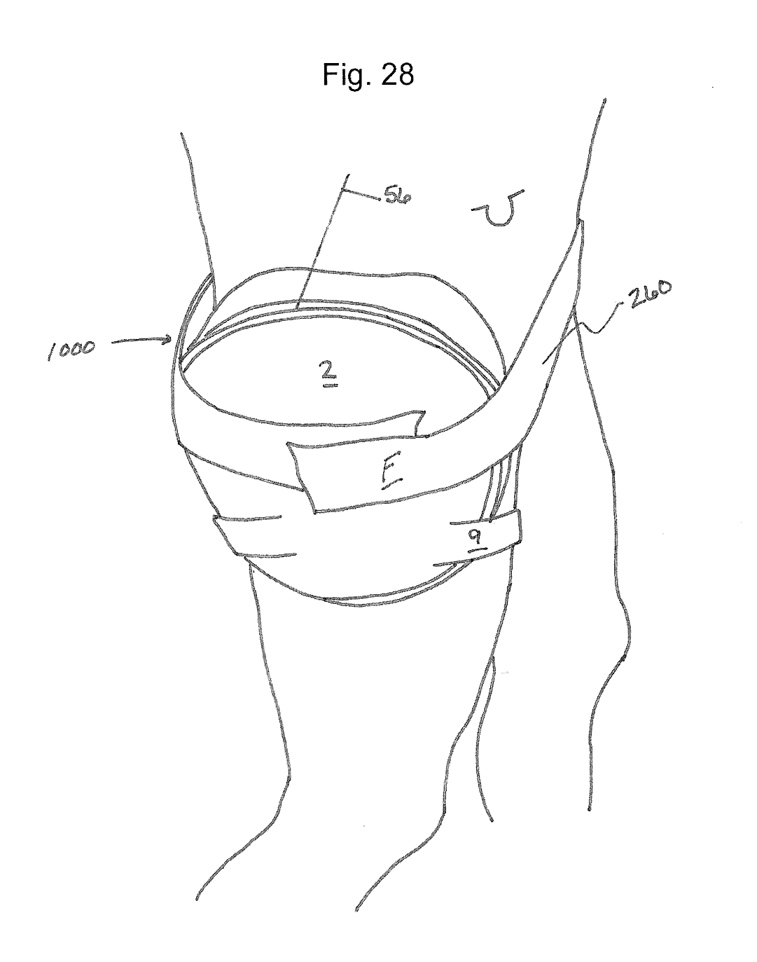

Referring next to FIG. 27 the user U has treated his left shoulder by daisy chaining pack 1000A to pack 1000B. The strap 9A with buckle 10A interlocks with buckle 10B and strap 9B. The long wrap 260 secures the package to the shoulder.

Referring next to FIG. 28 the user U has fastened the pack 1000 against his right hip using one strap 9 around his leg and one long wrap 260 around his waist.

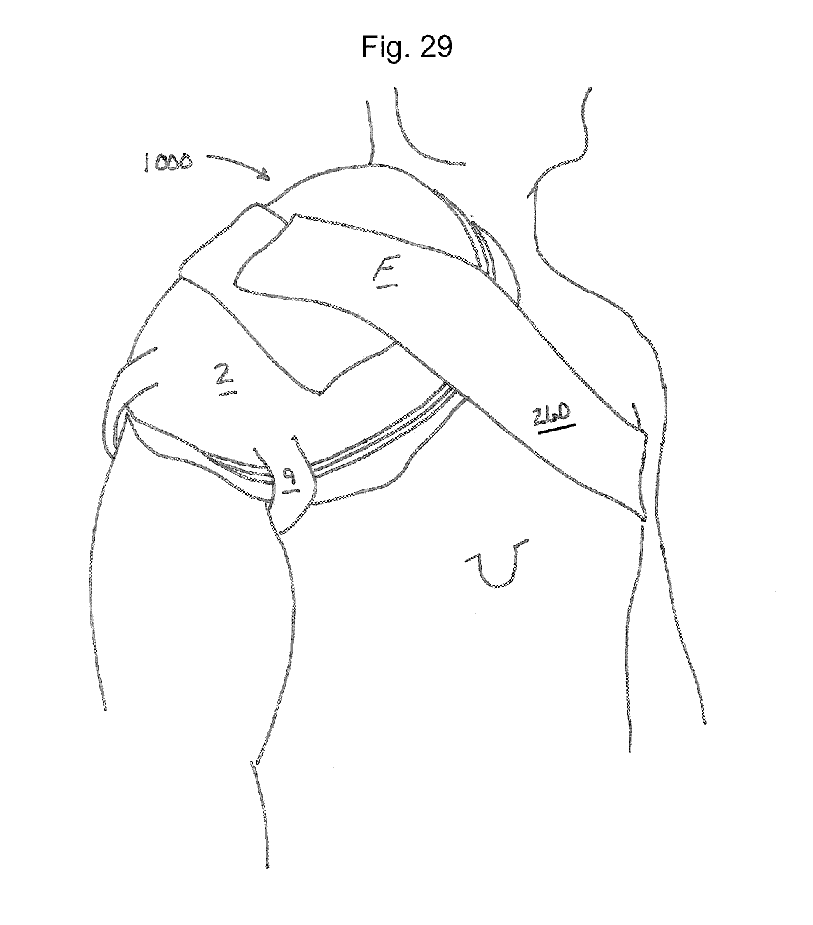

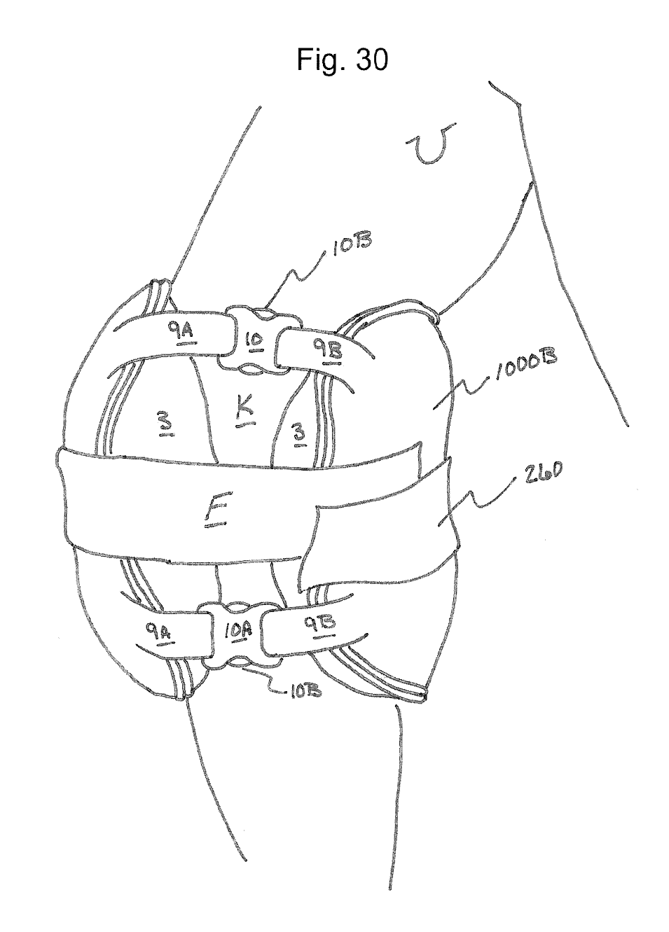

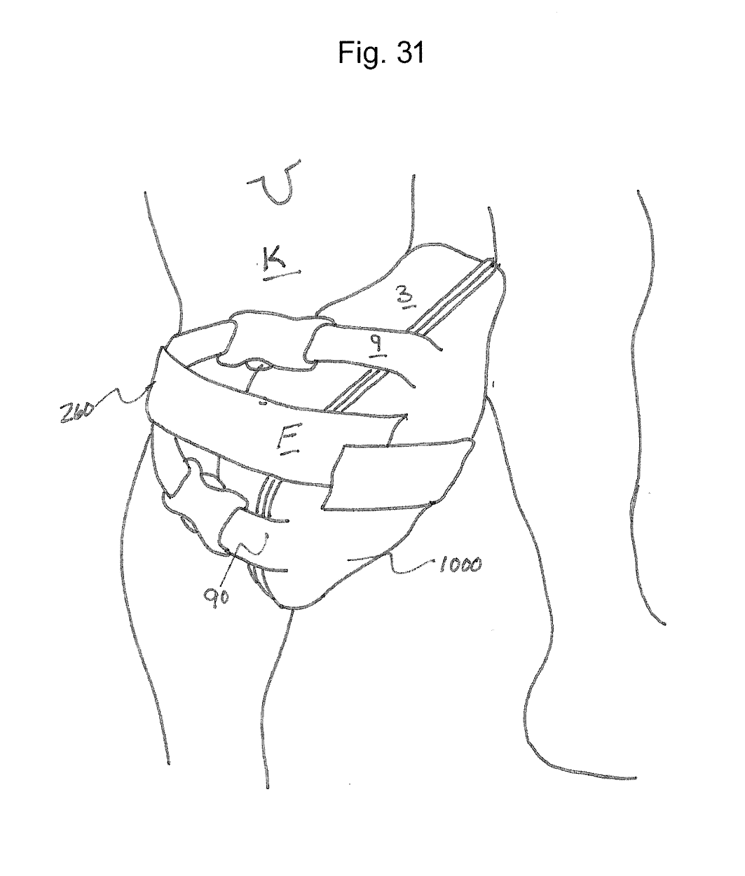

Referring next to FIG. 29 the user U has fastened the pack 1000 against his right shoulder using one strap 9 under his arm pit and the long wrap 260 around his upper torso.

Referring next to FIG. 30 the user U has secured a daisy chain pair of packs 1000A, 1000B to his left knee K, straps 9A and 9B are interconnected as are straps 90A, 90B.

Referring next to FIG. 31 the user U has secured pack 1000 to his right knee K using straps 9, 90 and long wrap 260.

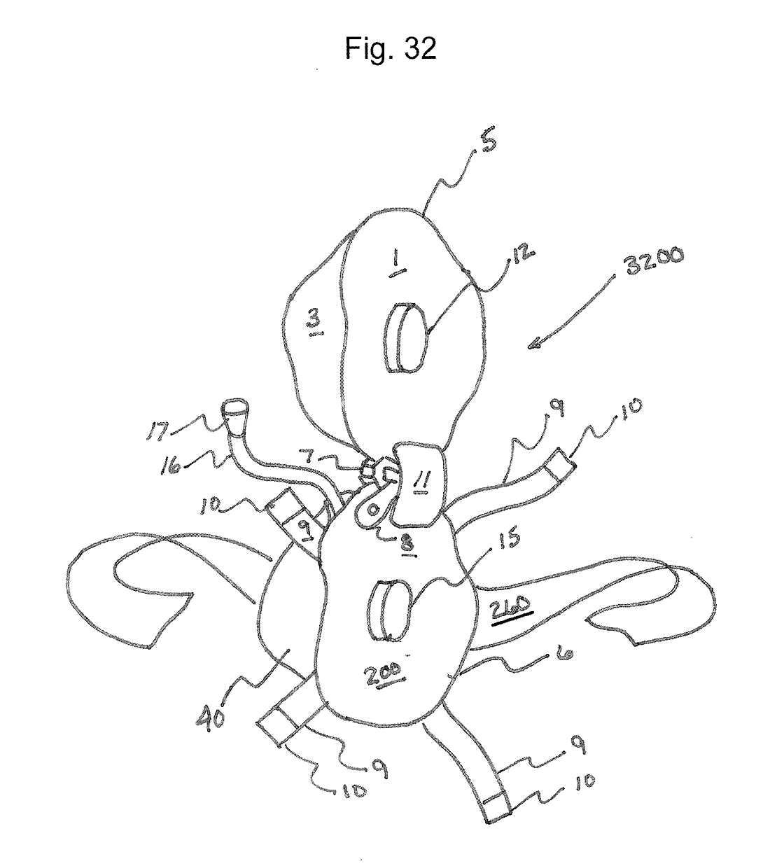

Referring next to FIGS. 32-35 a combo pack 3200 provides the same features as the pack 1000, but also incorporates a drinking water bottle 400 (hydration bladder) in the bottom clam shell half 200. A thin fabric 40 on the inside of the bottom half 200 of the clam shell secures the water bottle 400. A pocket P may cover the thin fabric 40. A hose 16 extends from the water bottle 400. A squeeze type muzzle 17 allows the user to drink on demand. A drinking water lid 15 is preferably a threaded cap. The water bottle 400 does not need to be removable from the combo pack 3200 since it can be placed under a faucet for filling.

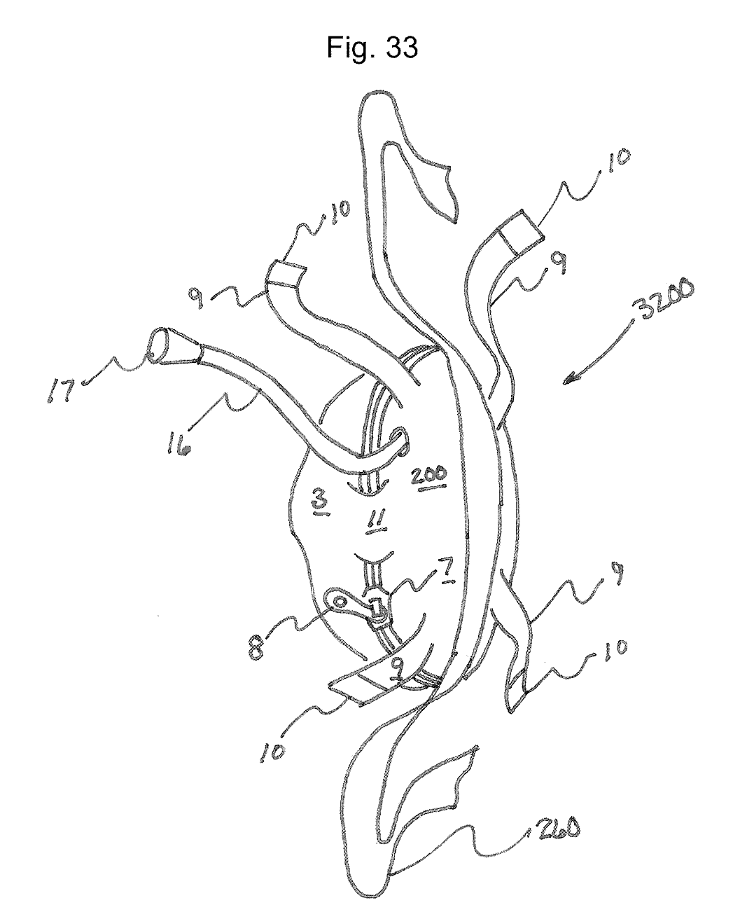

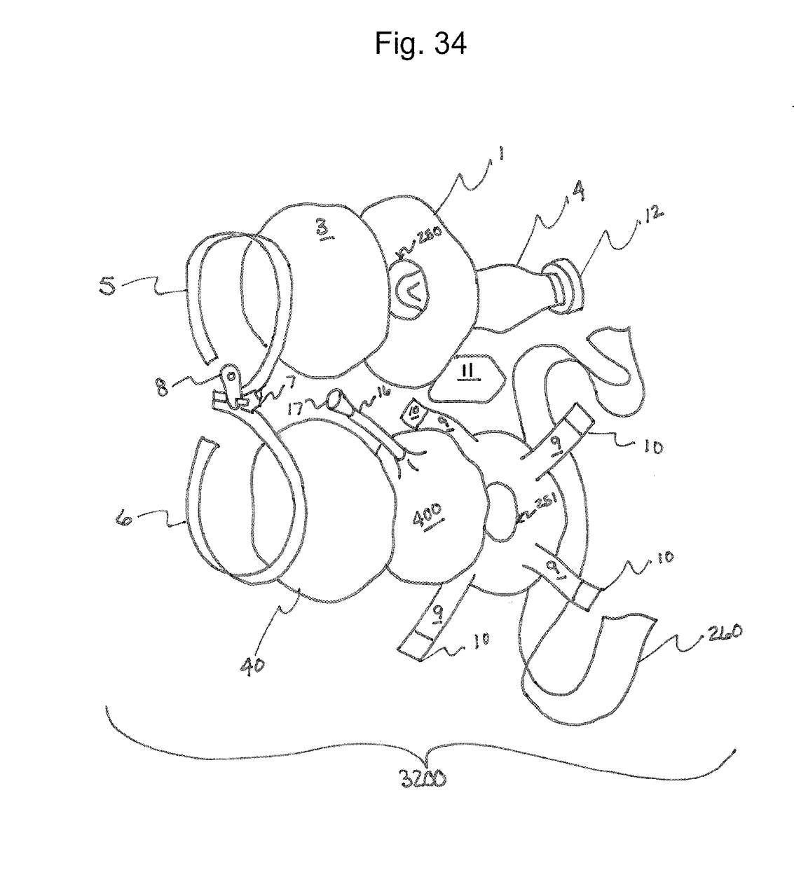

In FIG. 33 the pack 3200 is ready for application to an injury. In FIG. 34 a hole 251 in clam shell half 200 allows the lid 15 to connect to the water bottle 400.

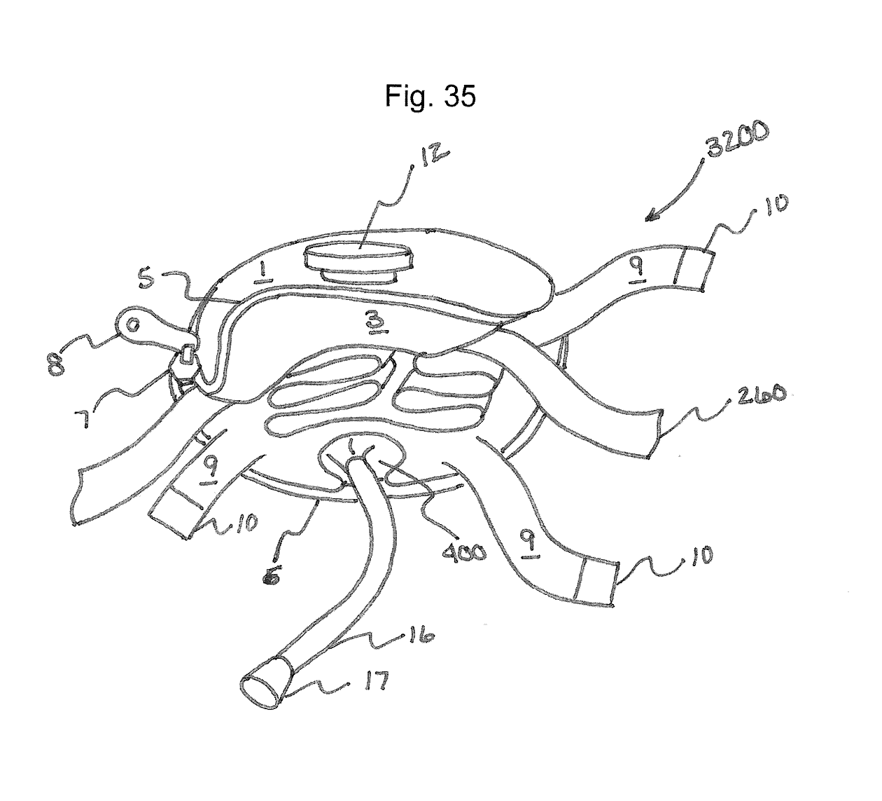

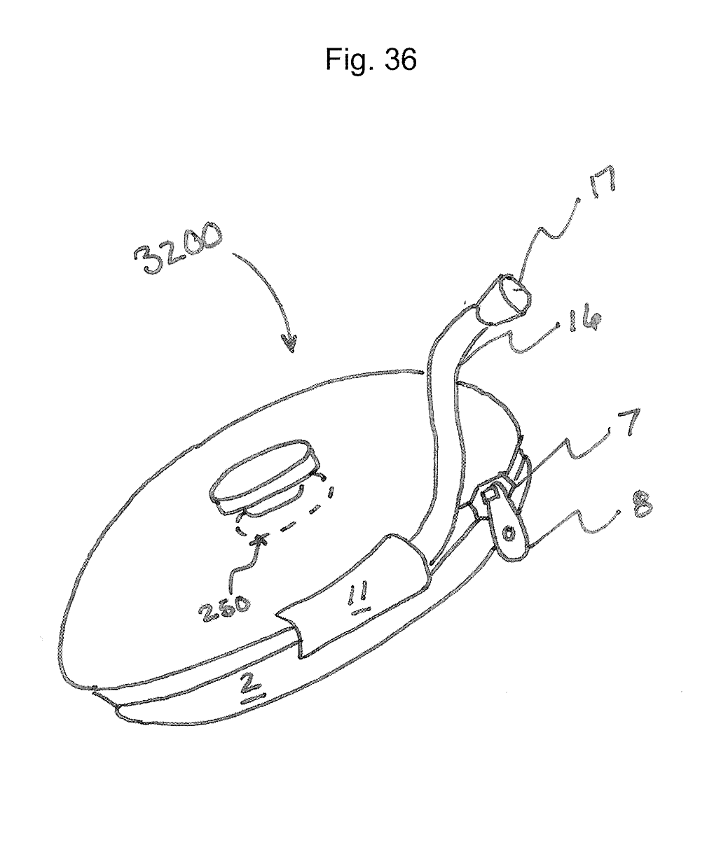

Referring next to FIG. 35 the user U is starting to transition the combo pack 3200 from the closed position. Nozzle 17 is available in any position.

Although the present invention has been described with reference to the disclosed embodiments, numerous modifications and variations can be made and still the result will come within the scope of the invention. No limitation with respect to the specific embodiments disclosed herein is intended or should be inferred. Each apparatus embodiment described herein has numerous equivalents.

I claim:

1. A thermal pack comprising:
a top and a bottom insulated cover, each cover being a mirror image of the other;
a connection means functioning to join the top to the bottom cover at a hinge point functioning to provide a closed mode which encircles a thermal pouch, an open mode which allows the top and the bottom covers to lie flat on a surface, and an application mode which reverses the top cover under the bottom cover and exposes the thermal pack for application to a human body part;
a closure means functioning to join the top to the bottom cover along a peripheral edge of each cover in either the closed mode or the application mode;
said thermal pouch secured to an inside surface of the top cover; and
at least one strap associated with the top or the bottom cover which enables affixing
the thermal pouch against the human body part.

2. The thermal pack of claim 1, wherein the connection means further comprises a fabric hinge.

3. The thermal pack of claim 2, wherein each of the top and bottom covers has a circular shape, and the thermal pack in the closed and application modes has a clam shell shape.

4. The thermal pack of claim 1, wherein the at least one strap further comprises a crisscrossed pair of straps each with a buckle and each with at least one permanent connection to the thermal pack.

5. The thermal pack of claim 4, wherein each crisscrossed strap is sewn at both ends to the bottom cover, and the bottom cover has an inside pocket to store the crisscrossed straps in.

6. The thermal pack of claim 5, wherein a long wrap is stored in the pocket which can be used to help secure the thermal pouch against the human body part.

7. The thermal pack of claim 1, wherein the top cover further comprises an inside layer to secure the thermal pouch against the top cover.

8. The thermal pack of claim 7, wherein the top cover further comprises an exit hole to allow insertion and retraction of the thermal pouch from the top cover.

9. The thermal pack of claim 1, wherein the closure means further comprises a zipper.

10. The thermal pack of claim 1, wherein the closure means further comprises a hook and loop fastener.

11. The thermal pack of claim 8, wherein the exit hole further comprises a plug to cover the thermal pouch.

12. The thermal pack of claim 1 further comprising a handle.

13. The thermal pack of claim 1, wherein the bottom cover further comprises a drinking water bladder.

14. The thermal pack of claim 13, wherein the drinking water bladder further comprises a hose.

15. A thermal pack comprising:
a clam shell pair of insulated covers having a hinged joint therebetween;
a zippered closure means joining the pair in either a closed transport mode or a reversed application mode;
a top member of the pair having an ice bag secured thereto;
a bottom member of the pair having a pair of straps secured thereto;
wherein the closed transport mode insulates the ice bag on the top and on the bottom; and
wherein reversing the top insulated cover to fold against the bottom insulated cover exposes the ice bag in an application mode ready to apply to an afflicted body part using the pair of straps.

16. The thermal pack of claim 15, wherein the top member of the pair further comprises a flexible sheet which secures the ice bag to an inside layer of the top member of the pair.

17. The thermal pack of claim 16, wherein the top member of the pair further comprises a hole for access to a lid of the ice bag.

18. The thermal pack of claim 15, wherein the ice bag further comprises a gel pack.

19. A combo pack comprising:
a top clam shell half comprising an insulated cover having an integral thermal pack;
a bottom clam shell half comprising an insulated cover having an integral thermal pack;
a bottom clam shell half comprising an insulated cover having an integral drinking water pouch;
a hinge joining the top to the bottom half;
a closed mode wherein the top and bottom halves are zippered shut to surround the thermal pack with insulated covers; and
a reverse mode wherein the top half is folded under the bottom half to expose the thermal pack for use, and the top and bottom halves are zippered together.

20. The combo pack of claim 19 further comprising a strap means functioning to secure the thermal pack against a body part.
